(19) United States
(12) Patent Application Publication
(10) Pub. No.: US 2013/0218081 A1
(43) Pub. Date: Aug. 22, 2013

(54) Pharmaceutical Delivery Device and Method for Providing Ocular Treatment

(71) Applicant: ForSight Vision4, Inc., (US)

(72) Inventor: Daniel B. Roth, Menlo Park, CA (US)

(73) Assignee: FORSIGHT VISION4, INC., Menlo Park, CA (US)

(21) Appl. No.: 13/849,445

(22) Filed: Mar. 22, 2013

Related U.S. Application Data
(63) Continuation of application No. 11/516,790, filed on Sep. 7, 2006, now abandoned.
(60) Provisional application No. 60/717,373, filed on Sep. 15, 2005.

(51) Int. Cl. A61F 9/00

(52) U.S. Cl. CPC A61F 9/0017
USPC 604/151; 604/244

(57) ABSTRACT

Disclosed herein is a novel pharmaceutical delivery device that provides controlled, sustained local delivery of a therapeutic agent of interest to a target tissue of interest, for example, the vitreous tissue of the eye, over an extended period of time.

### REFERENCE TO PRIORITY DOCUMENTS

This application is a continuation of co-pending U.S. patent application Ser. No. 11/516,790, filed Sep. 7, 2006, entitled “Pharmaceutical Delivery Device and Method for Providing Ocular Treatment,” which claims the benefit of priority of provisional application Ser. No. 60/717,373 filed Sep. 15, 2005. Priority of the aforementioned filing dates is hereby claimed and the disclosures of the applications are hereby incorporated by reference in their entirety.

### TECHNICAL FIELD

The present invention relates to the controlled, sustained, local delivery of a pharmaceutical of interest to a target tissue of interest, for example, the eye. More particularly, the present invention relates to a novel pharmaceutical delivery device useful for the treatment of ocular diseases and disorders, including, for example, macular degeneration, diabetic retinopathy and other pathologic conditions, through sustained release of therapeutic doses direct to specific ocular tissues.

### BACKGROUND

Developments in the treatment of retinal disease are expanding, particularly in the area of localized pharmaceutical drug delivery into the eye. New inhibitors of angiogenesis and vascular endothelial growth factor seem to be important in treating macular degeneration, diabetic retinopathy and

other conditions. These drugs cannot be effectively administered orally or intravenously without the risk of detrimental side effects. For this reason, it is advantageous to administer such drugs locally into the eye. Further, it is desirable to administer such drugs in a sustained release manner so that relatively small doses of the drug are exposed to the ocular system over an extended period of time.

Currently, most treatments are based upon intraocular injections into the eye, performed once a month or every 6-12 weeks. This becomes a tedious experience for patients and physicians alike and carries an increased risk associated with multiple intraocular injections (e.g., development of scar tissue, interference with vision, pain, infection, elevated intraocular pressure, etc.).

Within the last decade, several sustained release drug delivery devices have been disclosed that provide local delivery of a pharmaceutical directly to the eye. These devices generally include an inner drug core that contains an effective amount of a low-solubility, pharmaceutically active agent. The inner drug core includes a non-bio-erodible polymer layer that is permeable to the low solubility agent. The drug core is received within a holder. The holder is fabricated from an impermeable material, and includes one or more openings for passage of the pharmaceutically active agent to the surrounding ocular tissue. The holder holds the drug in the correct anatomic position during sustained release, and inhibits disintegration of the drug core while not significantly impairing the drug release rate.

In some delivery device embodiments, the pharmaceutical agent is placed within an impermeable holder. Strategically sized openings or “diffusion ports” are formed in the holder to permit controlled release of the agent into the ocular tissue. A semi-permeable housing is provided around the impermeable holder. Various dimensions and opening configurations have been proposed for such delivery devices.

The various models of drug delivery implants presently available carry certain disadvantages. For instance, the ganciclovir implant used to treat CMV retinitis is large and must be placed through an incision in the eyewall. This procedure essentially requires a partial vitrectomy. The drug is delivered over a 9 to 10 month period, after which the implant is ineffective. The implant must then be removed or another implant must be surgically implanted and sewn to the eyewall adjacent to it. This arrangement is expensive and inconvenient to the patient. In addition, as discussed above, repeated invasive procedures place the patient's vision at substantial risk.

A modified version of this implant is the recently FDA approved Retisert™, commercially available through Bausch and Lomb. With this implant, the steroid fluocinolone is released into the eye for approximately three years. The Retisert™ implant has the advantage of being smaller than the ganciclovir implant; nevertheless, it still requires surgical implantation and removal with a relatively large incision and vitreous prolapse.

The ganciclovir implant and the Retisert™ implant each use the concept of the semi-permeable membrane. An example of such a membrane material is polyvinyl alcohol, or “PVA.” The semi-permeable membrane allows the drug to enter the eye slowly over time at the acceptable dose. However, because the drug reservoir resides substantially within the tissue of the eye, a large incision must be made each time the device is implanted.

Another drug model involves impregnating a drug into a material similar to an absorbable suture. This noodle-like structure is then injected into the eye. Over a two to three month period of time, the polymer is degraded and the drug is released. This model has the advantage of providing a biodegradable insert; however, the implant only lasts a few months and therefore frequent repeated injections are required.

Another mechanism for delivering a drug into the ocular system involves placing the drug of interest into microspheres, whereby the drug is encapsulated in a lipid layer that slows down its absorption into the local tissues. This increases the survival (half life) of the drug in the eye, but cannot accomplish dosing for more than a few months (or less). Accordingly, as with the noodle device discussed above, frequent repeat dosing of the drug is required.

Therefore, a need exists in the art for a pharmaceutical delivery device that can be easily implanted through a less invasive procedure than with conventional implants. Further, a need exists for an implant wherein the drug reservoir resides substantially external to the sclera.

In addition, a need exists for a method that allows a drug to be delivered to the ocular structures slowly over time in adequate therapeutic doses without repeated procedures. The present addresses these and other needs in the art.

### SUMMARY

In view of the foregoing, it is an object of the present invention to provide a drug delivery device that provides controlled, sustained, and/or local delivery of a relevant therapeutic agent to target tissues, for example, ocular tissues, over an extended period of time. However, it will be understood by those skilled in the art that one or more aspects of this invention can meet certain objectives, while one or more other aspects can meet other objectives. Each objective may not apply equally, in all its respects, to every aspect and embodiment of this invention. As such, the following objects may be viewed in the alternative with respect to any one aspect of this invention.

Accordingly, it is an object of the present invention to provide an implantable pharmaceutical delivery device for controlled delivery of a therapeutic agent over an extended period of time to a target tissue of interest. In an illustrative embodiment, the device may be composed of a drug reservoir defined by (a) a hollow plate having an upper surface and a curved lower surface and (b) a hollow elongated stem projecting from the lower surface of the plate into the target tissue of interest, and (c) a means or mechanism for delivering the contents of the drug reservoir through the stem to target tissue of interest, examples of which include one or more diffusion ports and/or valved openings. Alternatively, the device may be fabricated, at least in part, from a semi-permeable material that allows for controlled diffusion of therapeutic agent across its barrier membrane into the tissue of interest.

In one preferred embodiment, the device is dimensioned and configured for use in an ocular environment; in this context, the elongated stem may be dimensioned to extend from the surface of the eye and through the choroid layer, such that at least the distal tip of the stem extends into the vitreous portion of the eye

In a particularly preferred embodiment, the present invention provides a pharmaceutical delivery device comprised of a curved hollow plate having a hollow elongated stem projecting from its concave underside, wherein the plate and stem have interior surfaces that define a hollow cavity for receiving a pharmaceutical formulation containing a therapeutic agent of interest.

In a further preferred embodiment, the pharmaceutical delivery device comprises a generally T-shaped implant having an upper curved plate and generally tubular stem depending therefrom, wherein the cavity formed by the plate and stem defines a generally umbrella or T-shaped drug reservoir. The underside of the curved plate is preferably specifically dimensioned to conform to the convex profile of a patient's eye and to reside substantially external to the patient's sclera, more preferably just below the conjunctival layer of the eye. The plate may optionally include a securing means, for example, one or more suture rings disposed at the edges or about the perimeter of the plate. Other securing mechanisms are contemplated herein and include, but are not limited, one or more layers of medical grade adhesive, one or more separate suture rings, one or more expandable sealing elements (e.g., an inflatable balloon disposed about or along the elongated stem), and the like.

It is a further object of the present invention to provide a method for delivering a therapeutic pharmaceutical agent to a target tissue of interest in a subject. The method preferably includes the step of implanting a pharmaceutical delivery device as described above in such a manner that the plate is not in direct contact with the target tissue while at least the distal tip of the stem extends into the target tissue. In this fashion, the majority of the drug reservoir is remote from the target tissue, preferably in a region that is accessible through substantially non-invasive means.

In one preferred embodiment, the present invention provides a method for delivering a therapeutic pharmaceutical agent to the ocular system of a subject that includes the steps of (a) forming an opening in a portion of a subject's eye that is disposed under the eyelid; (b) inserting the stem of the above-described pharmaceutical delivery device into the opening until the lower surface of the plate rests against the scleral layer of the eye; and securing the device in place.

In a further preferred embodiment, the present invention provides a method for delivering a pharmaceutical agent to the ocular system of a patient that optionally includes the steps of (a) performing a conjunctival peritomy of the eye of the patient, and then (b) forming a 23 to 25 gauge opening in the superotemporal or superonasal quadrant of the eye. While the exact location of the device is not particularly critical, it is preferable to situate the device away from the center of the eye, more preferably in the uppermost or lowermost portion of the sclera, i.e., that portion that is disposed under the upper or lower eyelid. A puncture incision is preferably made posterior to a surgical limbus in the middle of the quadrant of the eye. The stem of the pharmaceutical delivery device may then inserted into the puncture incision of the eye, permitting the eye to form a tight, self-sealing closure around the stem. The stem may be inserted until the underportion of the concave underside of the curved plate rests against the scleral layer of the eye. The plate may then secured to the sclera, for example, via sutures, medical grade adhesive or the like. The conjunctiva may then placed over the implant, covering it. The conjunctiva may sutured at the surgical limbus, protecting the implant from exposure.

These and other objects, features, benefits and advantages of the present invention will become more fully apparent when the following detailed description is read in conjunction with the accompanying figures, examples, data, and all reasonable inferences to be drawn therefrom.

### BRIEF DESCRIPTION OF THE DRAWINGS

So that the manner in which the above recited features of the present invention can be better understood, certain drawings, photographs and images are appended hereto. It is to be noted, however, that the appended figures illustrate only selected embodiments of the inventions and are therefore not to be considered limiting of scope, for the inventions may admit to other equally effective embodiments or applications.

FIG. 1A presents a cross-sectional view of a preferred embodiment of the pharmaceutical delivery device of the present invention.

FIG. 1B is a photograph depicting the side-view of another preferred embodiment of the pharmaceutical delivery device of the present invention.

FIG. 1C is a photograph depicting three prototypes of the pharmaceutical delivery device of the present invention, each with alternate optional stem configurations. The leftmost embodiment is provided with a tapered tip that facilitates atraumatic insertion. The central embodiment is provided with a rounded tip composed of a semipermeable material. The rightmost embodiment is provided with an open tip.

FIG. 2 is a cross-sectional view of an eye of a patient having received the pharmaceutical delivery device of the present invention depicted in FIG. 1A. The pharmaceutical delivery device is not shown in cross-section.

FIGS. 3A and 3B are photographs depicting side and front views, respectively, of an eye having received the pharmaceutical delivery device of the present invention depicted in FIG. 1B.

FIGS. 4A-4D are computer generated images that provide close-up views that more clearly depict the mating relationship between the curved underside of a pharmaceutical delivery system of the present invention and the curvature of the eye. A top view is provided in FIG. 4A, a perspective view in FIG. 4B, a front elevation view in FIG. 4C, and a side view in FIG. 4D. In these views, the optional opposing suture rings and their respective inner openings are more clearly visible.

### DETAILED DESCRIPTION

### Definitions

Unless otherwise defined, all technical and scientific terms used herein have the same meaning as commonly understood by one of ordinary skill in the art to which this invention belongs. In case of conflict, the present specification, including definitions, will control. Specifically, in the context of the present invention, the following definitions apply:

The term “proximal” refers to that end or portion of the drug delivery device that is anatomically located nearest to a point of reference, such as an origin or a point of attachment. Conversely, the term “distal” refers to that end or portion anatomically located far from a point of reference, such as an origin or a point of attachment. In the context of the present invention, the preferred point of reference is the surface of the eye. Accordingly, when positioned in the patient's eye, the portion of the elongated stem that is connected to the concave plate constitutes the “proximal” end of the stem while the free end of the stem constitutes the “distal” end of the stem. When properly positioned in the patient's eye, the free end of the stem is distal to the concave plate.

The term “concave” refers to a surface or boundary that curves inward, as to the inner surface of a sphere, or is hollowed or rounded inward like the inside of a bowl. Conversely, the term “convex” refers to a surface or boundary that curves outward, as the exterior of a sphere. Herein, the lower surface of the plate portion of the inventive device is preferably “concave” while the upper surface of the plate portion is preferably convex or dome-like.

The present invention makes reference to a semi-permeable membrane. In the context of the present invention, the semi-permeable membrane, also referred to a selectively permeable membrane, a partially permeable membrane or a differentially permeable membrane, is a membrane which will allow certain molecules or ions (for example, drug molecules) to pass through it by diffusion and occasionally specialized “facilitated diffusion”. The rate of passage depends on the pressure, concentration and temperature of the molecules or solutes on either side, as well as the permeability of the membrane to each solute.

The instant invention has both human medical and veterinary applications. Accordingly, the terms “subject” and “patient” are used interchangeably herein to refer to the person or animal being treated or examined. Exemplary animals include house pets, farm animals, and zoo animals. In a preferred embodiment, the subject is a mammal, more preferably a human.

The terms “pharmaceutical”, “medicament” and drug are used interchangeably to refer to any pharmaceutically active agent. The active agent may be any compound, composition of matter, or mixture thereof that can be delivered to the eye to produce a beneficial physiological or pharmacological result. The result may be systemic, though preferably it is specifically directed to treatment of the ocular system.

Non-limiting examples of such agents include: anti-viral agents such as ganciclovir, acyclovir, and AZT; antiglaucoma drugs such as beta-blockers; anti-angiogenesis agents such as metalloproteinase inhibitors, protein kinase C inhibitors, and endogenous angiogenesis inhibitors (e.g., angiostatin); anesthetics and pain killing agents; anti-inflammatory agents such as steroidal and non-steroidal anti-inflammatory agents; antiviral agents; antioxidants; antibiotics; antitumor agents such as tumor necrosis factors; anti-cataract agents; anti-glaucoma agents; insulin, cellular regeneration agents such as telomerase; steroidal compounds such as prednisolone, dexamethasone, and related compounds; low solubility steroids such as fluocinolone acetonide and related compounds; and antibiotics such as tetracycline, chlortetracycline, bacitracin, neomycin, polymyxin, gentamycin, vancomycin, amikkacin, ceftazidime, and erythromycin; growth factors, such as pigment epithelium-derived growth factor (PEDF), or inhibitors of growth factors, such as pegaptanib, ranibizumab, or bevacizumab. In the context of the illustrated embodiments, the preferred active agent is ranibizumab (sold under the tradename Lucentis™)

The pharmaceutical agent may be formulated as an injectable solution, for example an aqueous or non-aqueous sterile injection solution optionally containing additive ingredients such as excipients, isotonic agents, solubilizers, preservatives, buffers, bacteriostats and solutes which render the formulation isotonic with the blood of the intended recipient. Aqueous and non-aqueous sterile suspensions may further include suspending agents and thickening agents. Alternatively, the pharmaceutical may be formulated as an erodible solid, paste or viscous gel.

It should be understood that in addition to the ingredients particularly mentioned above, the formulations of this invention may include other agents conventional in the art having regard to the type of formulation in question. In addition, the formulation may be comprised of multiple pharmaceutical agents, alone or in combination with optional additives, excipients and inactive ingredients as needed.

### DESCRIPTION OF ILLUSTRATIVE EMBODIMENTS

The specific embodiments described herein are merely intended to illustrate the principles of the invention. Those skilled in the art will recognize that variations and modifications may be made to the embodiments without changing the principles of the invention herein disclosed. Accordingly, the accompanying figures, described in detail below, that depict aspects of the invention are in no way intended to limit the scope of the present invention.

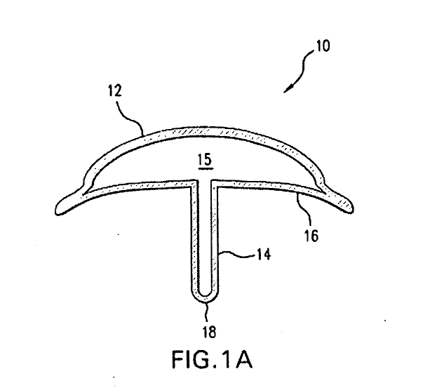

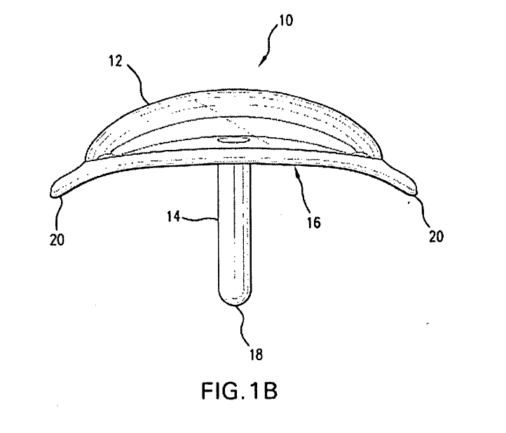

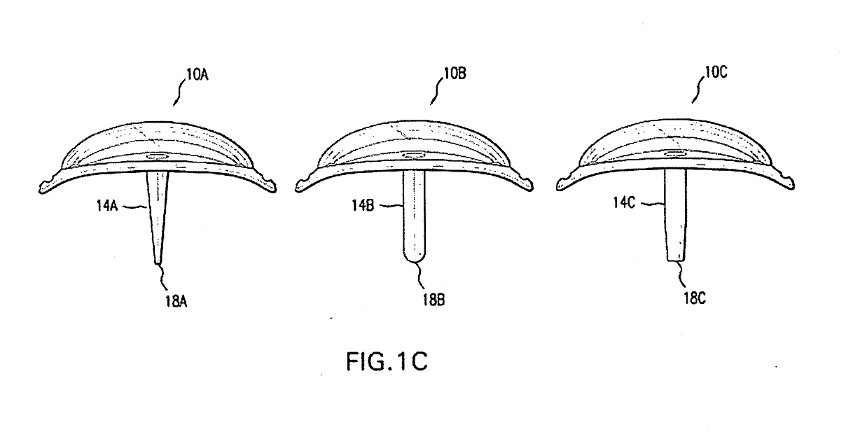

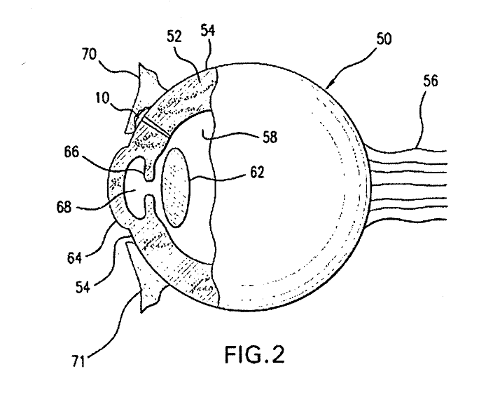

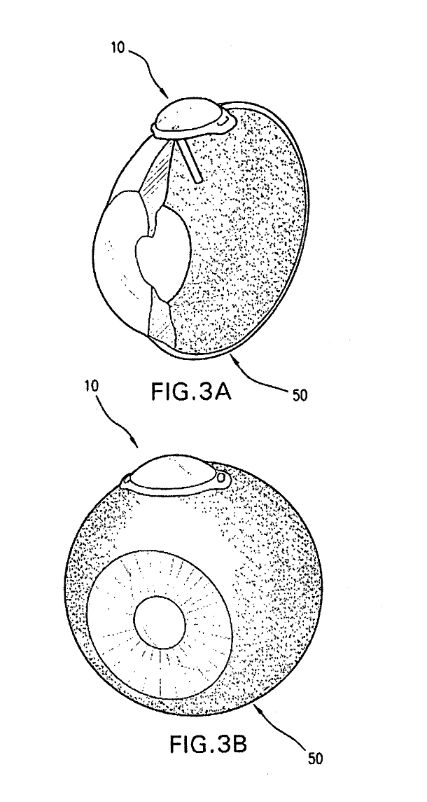

FIG. 1A presents a cross-sectional view of a pharmaceutical delivery device 10 of the present invention, in one embodiment. A side view of an analogous embodiment, including optional laterally opposed suture rings 20 and distal tip 18, is presented in FIG. 1B. The device 10 is configured to be implanted into the eye of a patient. While the device 10 may be implanted into the eye of any mammal, the device will be described in the context of a human as the patient herein. Such an eye is shown at 50 in FIGS. 2, 3A and 3B, which are described in detail below.

Photographs of prototypes having alternate stem configurations are provided in FIG. 10. In the leftmost embodiment, designated 10A, the stem 14A is provided with a tapered tip 18A that facilitates atraumatic insertion. In the central embodiment, 10B, the stem 14B is provided with a rounded tip 18B composed of a semipermeable material. In the rightmost embodiment, 10C, the stem 14C is provided with an open tip 18C. In the embodiment of 10C, the stem interior is preferably provided with a valve mechanism (not shown) that controls release of the drug contained within the reservoir.

The pharmaceutical delivery device 10 comprises a curved plate 12 and an elongated stem 14. The plate 12, generally a thin hollow member having curved upper and lower surfaces, more preferably a convex, dome-like upper surface and a concave lower surface, serves as an upper portion to the device 10, and is configured to rest upon the curved surface of the sclera (seen at 52 in FIG. 2), disposed between the sclera and the eyelid. The elongated stem 14, generally a thin, relatively cylindrical tube, defines a lower portion of the device that is received on the sclera 52 of the patient and extends through the choroid into the vitreous portion of the eye. The plate 12 and stem 14 are preferably integral to one another, being joined along an under portion 16 of the plate 12 or molded as a single unit. The interior surfaces of the hollow plate and stem define a drug reservoir for receiving a therapeutic agent of interest. In preferred embodiments, the stem is a generally tubular member that extends relatively normal to the plate so as to give the device a T configuration, the stem acting as a conduit for delivering the therapeutic agent from the drug reservoir, the majority of which resides in a location that is remote from the target tissue of interest, into the target tissue itself.

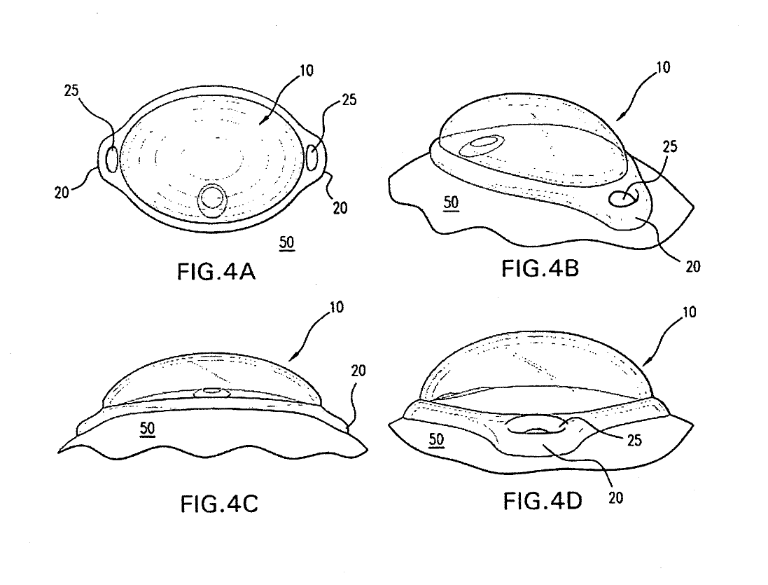

As best seen in FIG. 4, the lower surface of the device 10 preferably has a concave profile. This profile enables the lower surface of the device 10 to conform to the curvature of the eye 50. In one preferred embodiment, the plate 12 is oval or elliptical, having a major horizontal axis of approximately 3 to 25 mm, more preferably 5 to 20 mm, even more preferably 6 to 15 mm and a minor horizontal axis of approximately 3 to 15 mm, more preferably 4 to 12 mm, even more preferably 6 to 10 mm. However, it is readily apparent that while the curvature of the plate is important, neither the shape nor the precise dimension of the plate is particularly critical. Accordingly, other shapes (e.g., circular, rectangular, square, etc.) and sizes are contemplated herein.

The plate 12 is preferably fabricated from a soft, biocompatible material, for example a hard plastic or deformable silicone material. Preferably, the material is a smooth, tumble-polished, pliable silicone material. The thickness of the material from which the plate 12 is fabricated (i.e., the wall thickness of the plate) preferably ranges from 0.06 to 1.0 mm, more preferably 0.2 to 0.8 mm, even more preferably 0.4 to 0.6 mm.

The stem 14 defines a protrusion that is approximately 4 to 14 mm, more preferably 5 to 10 mm, even more preferably 6 to 8 mm in length. The length is measured from the underside 16 of the plate 12. While the precise length of the stem is not particularly critical, it is important that the stem be of length sufficient to penetrate the choroid and extend, at least at its distal tip, into the vitreous. To minimize trauma to the ocular tissues, the stem 14 preferably has an outer diameter on the order of 18 to 27 gauge, more preferably 20 to 25 gauge, even more preferably 23 to 25 gauge (or approximately 0.3 to 1.0 mm, more preferably 0.4 to 0.6 mm, even more preferably about 0.5 mm). In one preferred embodiment, the stem 14 is fabricated, at least in part, from a semi-permeable material that is inert, non-immunogenic and of the desired permeability. Preferably, polyvinyl alcohol, or PVA, is used. Other potentially suitable materials include ethylene vinyl acetate, silicone, polylactic acid, nylon, polypropylene, polycarbonate, cellulose, cellulose acetate, polyglycolic acid, cellulose esters and polyether sulfone. The thickness of the semi-permeable material from which the stem 14 is fabricated (i.e., the wall thickness of the hollow stem) preferably ranges from 0.06 to 1.0 mm, more preferably 0.2 to 0.8 mm, even more preferably 0.4 to 0.6 mm

Depending upon the desired rate of delivery, the semi-permeable membrane may constitute the entirety of the stem or, alternatively, only a select portion of the stem. For example, in one preferred embodiment, the distal tip of the stem is composed of a semi-permeable membrane while the remainder of the stem is composed on a impermeable biocompatible material, such as the pliable silicone discussed above. Alternatively, the stem may be entirely composed of an impermeable material yet provided with a series of mechanically provided openings or diffusion ports disposed about its periphery.

The use of semi-permeable materials to provide controlled release of medicament into an ocular area is disclosed in various patents, including:
U.S. Pat. No. 5,378,475 entitled “Sustained Release Drug Delivery Devices,” issued in 1995;
U.S. Pat. No. 5,902,598 entitled “Sustained Release Drug Delivery Devices,” issued in 1999; and
U.S. Pat. No. 6,375,972 entitled “Sustained Release Drug Delivery Devices, Methods of Use, and Methods of Manufacturing Thereof, issued in 2002,
   each of which is incorporated herein in its entirety by reference.

In an alternative embodiment, the stem may be open at the distal end yet provided with a one-way valve disposed along its length, preferably in proximity to the open distal tip, that controls the rate of drug delivery. An example of such an embodiment is depicted in FIG. 1C as 10C. Examples of suitable valve mechanism include, but are not limited to, single or double check valves, clapper and flap valves, globe valves, gate valves and the like, all of which are conventional in the art of drug delivery implants. In this and other embodiments, the device acts as a pump, wherein the act of blinking places pressure on the plate portion disposed on the scleral surface (e.g., the deformable dome-like upper surface of the plate portion) which, in turn, translates into an increase in fluid pressure within the reservoir that acts to open the one-way valve and deliver a metered dose of the contained within the reservoir to the vitreous tissues.

As shown and discussed more fully in connection with the embodiments depicted in FIGS. 1A-1C, the stem 14 is dimensioned to penetrate the sclera 52 of the patient's eye through the pars plana and into the vitreous cavity. In one aspect, an end of the stem 14 is fabricated from a layer of ethylene vinyl acetate or silicone.

As shown in FIG. 1A, the interior surfaces of the plate 12 and stem 14 together form a hollow reservoir 15 for receiving a pharmaceutical substance. The relative dimensions of the plate and stem are such that the bulk of the reservoir volume is remote from the target tissue of interest while the stem is in direct contact with the target tissue. For example, in the context of ocular applications, the majority of the reservoir volume (i.e., that defined by the interior of the plate) resides outside the eye, resting on the surface of the eye, while the tip of the stem projects into the vitreous cavity. The reservoir 15 within the plate portion 12 is defined by an inner diameter that, in a preferred embodiment, is approximately 18 to 21 mm along a major axis, 8 to 10 mm along a minor axis, and about 0.5 to 0.9 mm in height. The reservoir 15 within the stem portion 14 is defined by an inner diameter that is approximately 5 to 9 mm in length and 0.3 to 0.8 mm in width. The reservoir 15 receives the pharmaceutical formulation of interest. Preferably, the formulation takes the form of a viscous gel preparation that can easily migrate within the reservoir 15 and throughout the stem 14. However, other forms such as erodible pellets may be employed to enhance stability and predictability of release rate.

Preferably, sufficient medicament is placed in the reservoir 15 to provide one to three years of treatment. Various drugs may be used by modifying the drug delivery profile and/or the nature of the semi-permeable membrane on the protruding stem 14 that enters the eye 50. In one embodiment, 2 to 15 mg of fluocinolone acetonide is placed within the reservoir 15.

On opposing sides of the plate 12, one or more suture rings 20 may optionally be provided. Each ring 20 includes an inner opening 25 (best seen in FIG. 4) for receiving sutures. The rings 20 define smoothly rounded edges for compatibility with the ocular tissues. The inner diameter of each opening 25 is approximately 2-4 mm, while the outer diameter is preferably 4-8 mm.

The drug delivery device of the present invention may be held in place via alternative securing means. For example, the underside of the plate may be provided with one or more layers of medical grade adhesive. In another embodiment, the distal end of the stem may be enlarged or flared that hold the device across the choroids and prevents movement in the proximal direction. In yet another embodiment, the stem may be provided, at either end or along its perimeter, with an inflatable balloon that, once inflated, restricts relative movement of the device.

FIG. 2 is a cross-sectional view of an eye 50 having received the pharmaceutical delivery device 10 of FIG. 1A. The pharmaceutical delivery device 10 is not shown in cross-section. In this view, the device 10 has been implanted in an upper portion of the eye 50 and under the upper eyelid 70.

The eye 50 includes the sclera 52, commonly known as “the white of the eye.” Muscles (not shown, but including the rectus muscles) connect to the sclera 52 around the eye to control the eye's movements. The sclera 52 is received within the conjunctiva 54, which is a thin, transparent layer of tissue that covers the outer surface of the sclera 52. In FIG. 2, the stem 14 of the pharmaceutical delivery device 10 extends through the sclera 52 and into the vitreous cavity 58.

Connected to the sclera 52 at the back of the eye is the optic nerve 56. The optic nerve 56 transmits electrical impulses from the retina (not shown, but located along the back of the eye) to the brain.

Within the sclera 52 is the vitreous portion 58 of the eye 50. The vitreous 58 is a thick, transparent substance that fills the center of the eye 50. The vitreous is composed mainly of water and comprises about two-thirds of the eye's volume, giving it form and shape.

Other features of the eye 50 are shown in FIG. 2 for context. These include the cornea 64 (which is the transparent, dome-shaped window covering the front of the eye), the lower eyelid 71, the iris 66 (which defines the colored part of the eye and which controls light levels inside the eye), the anterior (or front) chamber 68, and the crystalline lens 62.

FIGS. 3A and 3B provide side and front views, respectively, of an eye 50 having received the pharmaceutical delivery device 10 of FIG. 1B. FIGS. 4A-4D provide a series of close-up views that more clearly depict the mating relationship between the curved underside of the pharmaceutical delivery device 10 and the curvature of the eye 50. In these views, the opposing rings 20 and their respective inner openings 25 are more clearly visible.

In order to implant the device 10 into the eye 50, a conjunctival peritemy is made using conventional surgical tools, such as a scalpel or scissors, for example Wescott™ type scissors. A 23 to 25 gauge opening is made preferably in the superotemporal or superonasal quadrant of the eye 50. Hemostasis of the scleral surface is achieved with diathermy. A puncture incision is made about 4 mm posterior to the surgical limbus in the middle of the quadrant with a sharp trocar. The trocar is designed to create an opening to accept the distal end 18 of the device 10. The stem 14 of the device 10 is inserted into the wound and forms a tight, self-sealing closure around the stem 14. The underportion 16 of the plate 12 meets the conjunctival layer 54. As noted, the plate 12 is configured to conform to the globe on the surface of the sclera 52.

The plate 12 may be sutured to the sclera 52 through the two or more opposing rings 20. More specifically, sutures may be sewn through the openings 25 in the rings 20 and into the sclera 52 adjacent to the rectus muscles. In one aspect, single interrupted 8-0 nylon sutures are used. The conjunctiva 54 is reflected over the device 10 and advanced to the limbus, completely covering the device 10. The conjunctiva 54 is then closed with 6-0 plain gut suture at each end.

With the stem 14 in place, the pharmaceutical material is able to be released through the semi-permeable membrane that makes up the stem 14. As the drug is released into the eye 50, treatment is provided at a controlled rate. Over time, the pharmaceutical within the reservoir 15 will be depleted. In this event, the reservoir 15 may be refilled by injecting new medicament through the plate 12 and into reservoir 15. Alternatively, the old device 10 may be surgically removed and a new pharmaceutical delivery device containing a new amount of drug may be inserted into the existing incision. The replacement procedure involves a minor procedure with minimal risk, meaning there is little likelihood that a new surgical incision into the sclera would be required; only a conjunctival peritomy.

If the puncture site needs to be permanently closed, this can be done with a single suture. A new overlying plate will seal the previous opening.

The device and method of the present invention constitute a substantial improvement over the presently available therapies in that it allow patients fewer visits with the doctor and the avoidance of monthly or bimonthly injections into the eye. In addition, the single relatively non-invasive procedure avoids the inherent risks of multiple procedures on an eye.

The principles of this invention have been described in connection with specific examples and preferred embodiments. However, it should be clearly understood that these descriptions are added only by way of example and are not intended to limit, in any way, the scope of the invention, which is defined by the pending claims and their equivalents. In other words, while application to the eye is described in particular detail, it will be apparent to those skilled in the art that the drug delivery device of the present invention may be applicable to other organs and systems, including, for example, intraarticular or intrathecal drug delivery, for the treatment of conditions that benefit from sustained, controlled and/or local delivery of therapeutically relevant agents (e.g., management of recurrent and/or chronic pain and/or inflammation).

For example, the drug delivery device of the present invention finds utility in the context of acute, chronic and/or intractable pain management and palliative care, e.g., in the treatment of terminal cancer patients and such. The drug delivery device of the present invention also finds utility in the treatment of certain inflammatory conditions, such as bursitis, tendonitis and arthritis, such treatment often involving repeated local injections of corticosteroids (e.g., cortisone) to a remote target tissue. Similarly, epidural steroid injections are often used in the context of rehabilitation (e.g., to provide pain relief to enable patients to progress with activities that are critical to rehabilitating the lower back and to prevent or minimize future episodes of lower back pain) or pain management (e.g., to provide a non-surgical option for the treatment of conditions, such as lumbar disc herniation, degenerative disc disease, lumbar spine stenosis, and the like, that are associated with severe acute or chronic lower back and/or leg pain). While a single injection can provide up to months of relief, inflammation and the associated pain frequently recurs and requires one or more subsequent injections to afford relief. It is well-established that intrathecal and intraarticular injections are not only painful but also are associated with a number of substantial risks, including infection, bleeding, nerve damage, and, in the case of epidural injections, dural puncture. Accordingly, the device of the present invention finds advantageous utility in this context. In particular, the pharmaceutical delivery device of the present invention can be affixed or implanted, preferably by means of a relatively minor and/or non-invasive procedure, in a manner such that the bulk of the device (e.g., the plate portion) is remote, external and/or superficial to the target tissue (e.g., subcutaneously implanted) while at least the distal tip of the stem extends into the target tissue itself (e.g., the bursa, tendon, joint, epidural space, etc.). In this manner, the device of the present invention can deliver a metered dose of medicament directly to the target tissue of interest over an extended period of time and thereby provide continuous relief while avoiding the need for repeated, often times dangerous and/or painful, procedures.

The disclosure of each publication, patent or patent application mentioned in this specification is specifically incorporated by reference herein in its entirety.

What is claimed is:

1. A device for treating an ophthalmic condition comprising:
a reservoir having a convex upper surface configured to be penetrated for refilling of the reservoir after implantation in an eye and a concave lower surface;
a conduit in fluid communication with the reservoir and coupled to the concave lower surface opposite the convex upper surface; and
a valve mechanism disposed within the conduit, wherein the valve mechanism controls fluid delivery through the conduit and prevents backflow,
wherein the device is configured to be implanted with the lower surface placed adjacent to a sclera of an eye and the conduit inserted into the eye through the sclera to facilitate delivery of a therapeutic agent from within the reservoir into the eye.

2. The device of claim 1, wherein the reservoir has a major horizontal axis that is approximately 5 mm, a minor horizontal axis that is approximately 3 mmm and a height between the convex upper surface and the concave lower surface that is between 0.5 mm and 0.9 mm.

3. The device of claim 1, wherein the therapeutic agent is selected from the group consisting of an anti-viral agent, ganciclovir, acyclovir, AZT, a beta-blocker, anti-angiogenesis agent, a metalloproteinase inhibitor, a protein kinase C inhibitor, an endogenous angiogenesis inhibitor, angiostatin, an anesthetic or pain killing agent, a steroidal or non-steroidal anti-inflammatory agent, an antioxidant, an antibiotic, antitumor agent, a tumor necrosis factor, an anti-cataract agent, an anti-glaucoma agent, insulin, a cellular regeneration agent, telomerase, a steroidal compound, prednisolone, dexamethasone, a growth factor inhibitor, fluocinolone acetonide, tetracycline, chlortetracycline, bacitracin, neomycin, polymyxin, gentamycin, vancomycin, amikkacin, ceftazidime, erythromycin, growth factors, pigment epithelium-derived growth factor (PEDF), inhibitors of growth factors, pegaptanib, ranibizumab, and bevacizumab.

4. The device of claim 1, wherein the therapeutic agent is pegaptanib, ranibizumab, or bevacizumab.

5. The device of claim 1, wherein the conduit directs the therapeutic agent to a target tissue.

6. The device of claim 1, wherein the valve mechanism is a one-way valve.

7. The device of claim 1, wherein the conduit further comprises an at least partially permeable material.

8. The device of claim 1, wherein the therapeutic agent is delivered through the conduit by pumping action.
